(19) United States
(12) Patent Application Publication
(10) Pub. No.: US 2008/0039670 A1
(43) Pub. Date: Feb. 14, 2008

(54) Methanol-Water Mixtures in Olefin Production Via Oxygenate Conversion

(76) Inventors: Lawrence W. Miller, Palatine, IL (US); Sterling T. Miller, Rolling Meadows, IL (US); Andrea A. Bozzano, Northbrook, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/463,757

(22) Filed: Aug. 10, 2006

(51) Int. Cl. C07C 1/00

(52) U.S. Cl. 585/639

(57) ABSTRACT

Processing schemes and arrangements are provided for producing light olefins from an oxygenate-containing feedstock and using methanol-water mixtures to recover oxygenates such as for further processing to form additional light olefins.

### BACKGROUND OF THE INVENTION

This invention relates generally to the conversion of oxygenates to olefins and, more particularly, to light olefins using methanol-water mixtures.

A major portion of the worldwide petrochemical industry is involved with the production of light olefin materials and the subsequent use of such light olefin materials in the production of numerous important chemical products. The production and use of light olefin materials may involve various well-known chemical reactions including, for example, polymerization, oligomerization, and alkylation reactions. Light olefins generally include ethylene, propylene and mixtures thereof. These light olefins are essential building blocks used in the modern petrochemical and chemical industries. A major source for light olefins in present day refining is the steam cracking of petroleum feeds. For various reasons including geographical, economic, political and diminished supply considerations, the art has long sought sources other than petroleum for the massive quantities of raw materials that are needed to adequately supply the demand for these light olefin materials.

The search for alternative materials for light olefin production has led to the use of oxygenates such as alcohols and, more particularly, to the use of methanol, ethanol, and higher alcohols or their derivatives or other oxygenates such as dimethyl ether, diethyl ether, etc., for example. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

Such processing, wherein an oxygenate-containing feed is primarily methanol or a methanol-water combination (including crude methanol), typically results in the conversion of methanol to light olefins (mostly ethylene and propylene) is commonly referred to as Methanol-to-Olefin (MTO) processing. Dimethyl ether (DME) is a typical byproduct or intermediate of such reaction processing. Consequently, small amounts of DME as well as other oxygenate byproducts that may be produced as a result of such processing are commonly discharged from the oxygenate conversion reactor together with the desired light olefin products for downstream product recovery.

DME and other oxygenate byproducts, if recovered, can desirably be recycled to the oxygenate conversion reactor for conversion to valuable olefin products, preferably, light olefins. Such recovery and subsequent processing, however, have conventionally been subject to various limitations and restrictions.

For example, as the boiling point of DME is very near the boiling point of propylene, the separation and recovery of DME from a propylene-containing stream can be problematic for conventional techniques such as conventional fractionation. Moreover, as DME is typically a catalyst poison for catalyst materials in polypropylene units, DME desirably is removed from the propylene feedstock to a polypropylene unit.

Alternatively, DME can be scrubbed from the oxygenate conversion reactor effluent vapors using methanol. Methanol, however, also commonly absorbs significant and undesirable quantities of olefins, particularly propylene. Recycle of such absorbed olefins will undesirably and unnecessarily increase the capital cost of upstream equipment and also increase the utilities required for proper operation of the system.

In addition, while MTO processing converts methanol to ethylene and propylene as its primary products, the process also has some selectivity to heavy olefins, i.e., C₄+ olefins, which are commonly of relatively low commercial value. The amounts of light olefins resulting from MTO processing, however, can be further increased by reacting, i.e., cracking, heavier hydrocarbon products, particularly heavier olefins such as C₄ and C₅ olefins, to light olefins. For example, commonly assigned, U.S. Pat. No. 5,914,433 to Marker, the entire disclosure of which is incorporated herein by reference, discloses a process for the production of light olefins comprising olefins having from 2 to 4 carbon atoms per molecule from an oxygenate feedstock. The process comprises passing the oxygenate feedstock to an oxygenate conversion zone containing a metal aluminophosphate catalyst to produce a light olefin stream. A propylene stream and/or mixed butylene is fractionated from said light olefin stream and cracked to enhance the yield of ethylene and propylene products. This combination of light olefin product and propylene and butylene cracking in a riser cracking zone or a separate cracking zone provides flexibility to the process which overcomes the equilibrium limitations of the aluminophosphate catalyst. In addition, the invention provides the advantage of extended catalyst life and greater catalyst stability in the oxygenate conversion zone.

As catalysts commonly employed in olefin cracking processes can be sensitive to hydrothermal damage, it is generally desirable to limit the amount or concentration of water in the feed to such a process. Moreover, as it has been discovered that oxygenate species present in olefin cracking process feeds can convert to water, appropriately limiting the amount or concentration of such species in the feed can also be important.

In view thereof, there is an ongoing need and demand for improved processing and systems for the conversion of oxygenates to olefins and, more particularly, for such processing and systems such as to result in improved oxygenate recovery from appropriate product streams such as to facilitate processing of either or both such recovered oxygenate materials for conversion processing such as to result in added or increased olefin production and processing of heavier olefin products such as to result in added or increased light olefin production.

### SUMMARY OF THE INVENTION

A general object of the invention is to provide or result in improved processing of an oxygenate-containing feedstock to light olefins.

A more specific objective of the invention is to overcome one or more of the problems described above.

The general object of the invention can be attained, at least in part, through a process for producing light olefins from a methanol-containing feedstock. In accordance with one preferred embodiment, such a process involves contacting the methanol-containing feedstock in an oxygenate conversion reactor with an oxygenate conversion catalyst and at reaction conditions effective to convert the methanol-containing feedstock to an oxygenate conversion product stream comprising light olefins, C₄+ hydrocarbons and oxygenates. At least a liquid portion of the oxygenate conversion product stream is contacted in an absorber with a solvent mixture comprising at least methanol and water. The solvent mixture is effective to absorb a significant portion of the oxygenates from the contacted portion of the oxygenate conversion product stream. At least a portion of the oxygenates absorbed from the contacted portion of the oxygenate conversion product stream is fed to the oxygenate conversion reactor for contact with the oxygenate conversion catalyst and at reaction conditions effective to convert at least a portion of the oxygenates to oxygenate conversion products.

The prior art generally fails to provide processing of oxygenates, particularly methanol, to olefins, such as to result in an increase in the relative amount of light olefins, and which processing is one or more as simple, effective, as economic as may be desired.

In accordance with one embodiment, the oxygenate conversion product stream comprises the oxygenate dimethyl ether. At least a portion of the oxygenate conversion product stream is contacted in an absorber with a solvent mixture comprising at least methanol and water effective to absorb and recover a significant portion of the dimethyl ether from the oxygenate conversion product stream. At least a portion of the dimethyl ether absorbed from the conversion product stream is subsequently introduced into the oxygenate conversion reactor and reacted to form additional oxygenate conversion products.

In accordance with another embodiment, the process involves treating the oxygenate conversion product stream in a gas concentration system to recover light olefins and to form a C₄+ hydrocarbon stream also containing oxygenates. The C₄+ hydrocarbon stream also containing oxygenates may desirably comprise the portion of the oxygenate conversion product stream contacting with the solvent mixture in the absorber and wherein as a result of said contacting a C₄+ hydrocarbon stream having a reduced oxygenate content is formed. At least a portion of the C₄+ hydrocarbon stream having a reduced oxygenate content contacts with an olefin cracking catalyst in an olefin cracking reactor at reaction conditions effective to convert C₄ and C₅ olefins therein contained to a cracked olefins effluent stream comprising light olefins.

A system for converting methanol to light olefins is also provided. In accordance with one preferred embodiment, such as system includes a reactor for contacting a methanol-containing feedstream with catalyst and converting the methanol-containing feedstream to an oxygenate conversion product stream comprising light olefins, C₄+ hydrocarbons and oxygenates. The system also includes an absorber wherein at least a portion of the oxygenate conversion product stream contacts a solvent mixture comprising at least methanol and water. The solvent mixture is effective to absorb a significant portion of the oxygenates from the contacted portion of the oxygenate conversion product stream. A first return line is provided wherein at least a portion of the oxygenates absorbed from the contacted portion of the oxygenate conversion product stream is introduced to the oxygenate conversion reactor for contact with the oxygenate conversion catalyst and at reaction conditions effective to convert at least a portion of the oxygenates to oxygenate conversion products.

As used herein, references to “light olefins” are to be understood to generally refer to C₂ and C₃ olefins, i.e., ethylene and propylene.

“Oxygenates” are hydrocarbons that contain one or more oxygen atoms. Typical oxygenates include alcohols and ethers, for example.

References to “Cₓ hydrocarbon” are to be understood to refer to hydrocarbon molecules having the number of carbon atoms represented by the subscript “x”. Similarly, the term “Cₓ-containing stream” refers to a stream that contains Cₓ hydrocarbon. The term “Cₓ+ hydrocarbons” refers to hydrocarbon molecules having the number of carbon atoms represented by the subscript “x” or greater. For example, “C₄+ hydrocarbons” include C₄, C₅ and higher carbon number hydrocarbons.

References to “absorption”, “absorbing”, “absorber” and the like when used herein in reference to the treatment of a process stream to effect removal of oxygenates are to be understood as also encompassing extraction processing and the like.

Other objects and advantages will be apparent to those skilled in the art from the following detailed description taken in conjunction with the appended claims and drawings.

### BRIEF DESCRIPTION OF THE DRAWINGS

FIG. 1 is a simplified schematic diagram of a process for the production of olefins and, more specifically, a process for the production of olefins, particularly light olefins; via oxygenate conversion processing in accordance with one embodiment.

FIG. 2 is a simplified schematic diagram of a section of a treatment and hydrocarbon recovery zone in accordance with one embodiment.

FIG. 3 a simplified schematic diagram of a pre-olefin cracking C₄+ hydrocarbon stream treatment zone in accordance with one embodiment.

FIG. 4 a simplified schematic diagram of a pre-olefin cracking C₄+ hydrocarbon stream treatment zone in accordance with another embodiment.

FIG. 5 a simplified schematic diagram of a processing scheme in accordance with another embodiment.

### DETAILED DESCRIPTION OF THE INVENTION

As described above, an oxygenate-containing feedstock can be converted to olefins and, more particularly, to light olefins via a catalytic reaction such as in an oxygenate conversion reactor. Heavier hydrocarbons (e.g., C₄+ hydrocarbons) formed during such processing can be subsequently cracked to increase the light olefins (e.g., C₂ and C₃ olefins) produced or resulting from the overall process.

As described in greater detail below, prior to such heavier hydrocarbon cracking processing, the process stream can desirably be processed by contacting at least a portion of the oxygenate conversion product stream in an absorber with a solvent mixture including at least methanol and water, as such a solvent mixture has been found to be particularly effective in the liquid-liquid absorption or liquid-liquid contact and removal of a significant portion of the oxygenates from the contacted portion of the oxygenate conversion product stream without detrimentally also absorbing significant quantities of olefins also present in the product stream.

In certain preferred embodiments, the solvent mixture employed in such absorption or removal may desirably consist essentially of methanol and water.

While the broader practice of the invention is not necessarily limited by or to the removal of specific quantities or proportions of the oxygenates present in such a contacted portion of the oxygenate conversion product stream, references to a “significant portion” as used herein in reference to oxygenates absorbed or removed from the contacted portion of the oxygenate conversion product stream are to be understood to generally constitute at least about 70% of the oxygenates present therein. In accordance with certain preferred embodiments, references to a “significant portion” as used herein in reference to oxygenates absorbed or removed from the contacted portion of the oxygenate conversion product stream are to be understood to generally constitute at least about 80% of the oxygenates present therein. In accordance with other certain preferred embodiments, references to a “significant portion” as used herein in reference to oxygenates absorbed or removed from the contacted portion of the oxygenate conversion product stream are to be understood to generally constitute at least about 95% of the oxygenates present therein.

At least a portion of the oxygenates absorbed from the contacted portion of the oxygenate conversion product stream can subsequently be processed such as via the oxygenate conversion reactor to form additional oxygenate conversion products.

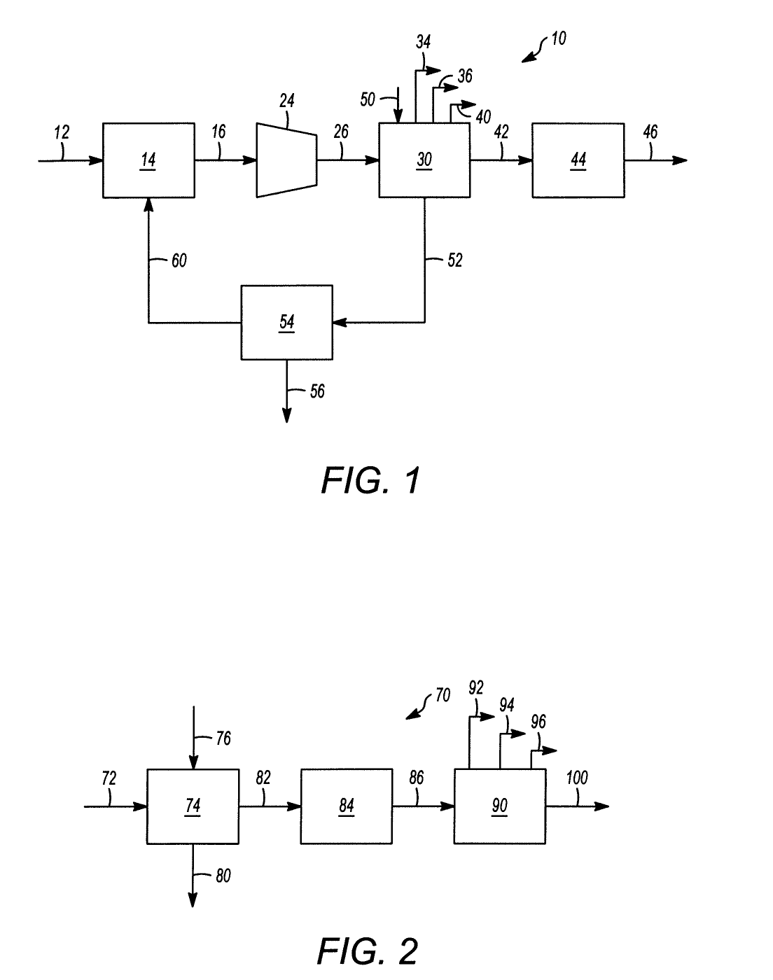

As will be appreciated, such processing may be embodied in a variety of processing arrangements. As representative, FIG. 1 illustrates a simplified schematic process flow diagram for a process scheme, generally designated by the reference numeral 10, for the production of olefins, particularly light olefins, via oxygenate (e.g., methanol) conversion processing. It is to be understood that no unnecessary limitation to the scope of the claims which follow is intended by the following description. Those skilled in the art and guided by the teachings herein provided will recognize and appreciate that the illustrated process flow diagram has been simplified by the elimination of various usual or customary pieces of process equipment including some heat exchangers, process control systems, pumps, fractionation systems, and the like. It may also be discerned that the process flow depicted in the FIG. 1 may be modified in many aspects without departing from the basic overall concept of the invention.

An oxygenate-containing feedstock, shown as a line 12 and such as generally composed of methanol and, if desired, one or more additional light oxygenates such as one or more of ethanol, dimethyl ether (“DME”), diethyl ether, or mixtures thereof, is introduced into an oxygenate conversion reactor section 14 wherein the oxygenate-containing feedstock contacts with an oxygenate conversion catalyst at reaction conditions effective to convert the oxygenate-containing feedstock to an oxygenate conversion product stream, shown as a line 16, such as comprising light olefins and C₄+ hydrocarbons, in a manner as is known in the art, such as, for example, utilizing a fluidized bed reactor. Those skilled in the art and guided by the teachings herein provided will appreciate that such an oxygenate conversion product stream may also typically include or contain additional components or materials such as either or both fuel gas hydrocarbons and oxygenate species including unreacted feed oxygenates, e.g., methanol, as well as oxygenate byproduct species such as dimethyl ether and such as formed in situ as a byproduct or intermediate of the desired methanol-to-oxygenate conversion process as well varying or trace quantities of other oxygenate byproducts. In practice, such other oxygenate byproducts are typically in the nature of light alcohols, aldehydes and ketones.

As will be appreciated by those skilled in the art and guided by the teachings herein provided, such suitable such oxygenate-containing feedstock may constitute commercial grade methanol, crude methanol or various combinations thereof. Crude methanol may be an unrefined product from a methanol synthesis unit. Moreover, in the interest of factors such as improved catalyst stability, embodiments utilizing higher purity methanol feeds may be preferred. Thus, suitable feeds may comprise methanol or a methanol and water blend, with possible such feeds having a methanol content of between about 65% and about 100% by weight, preferably a methanol content of between about 80% and about 100% by weight and, in accordance one preferred embodiment, a methanol content of between about 95% and about 100% by weight.

The oxygenate conversion product stream of the line 16 is appropriately processed through a compressor section 24, such as may be desirably composed of a plurality of compression stages. Additional processing steps may intervene between the reactor section 14 and the compressor section 24 to cool and/or reduce the volume or concentration of byproduct in the product stream in line 16. The resulting compressed oxygenate conversion product stream, shown as a line 26, such as after appropriate cooling (not shown) is introduced into an appropriate treatment and hydrocarbon recovery zone 30. As described in greater detail below and in accordance with one preferred embodiment (see FIG. 2, for example), such a treatment and hydrocarbon recovery zone may desirably include one or more unit operations whereby the oxygenate conversion product stream can be treated, such as via a liquid-liquid absorption, extraction or contact and removal with a methanol and water solvent mixture to remove and desirably recover selected species, such as oxygenates, such as DME. Alternatively, if desired, the oxygenate conversion product stream can be processed through a conventional oxygenate recovery scheme such as involving separate contacting such as in a wash column with first either a methanol or low purity water (e.g., recycle water) wash stream followed with a subsequent water wash stream such as in a water wash column such as to recover remaining methanol or with a high purity water to recover remaining oxygenate materials.

The treatment and hydrocarbon recovery zone 30 may desirably include a hydrocarbon recovery section such as generally composed of a gas concentration system. Through such hydrocarbon recovery section processing, the oxygenate conversion product stream materials can be processed to at least separately concentrate and recover a fuel gas stream 34, an ethylene stream 36, a propylene stream 40 and a mixed C₄+ hydrocarbon stream 42, such as generally composed of butylenes and heavier hydrocarbons.

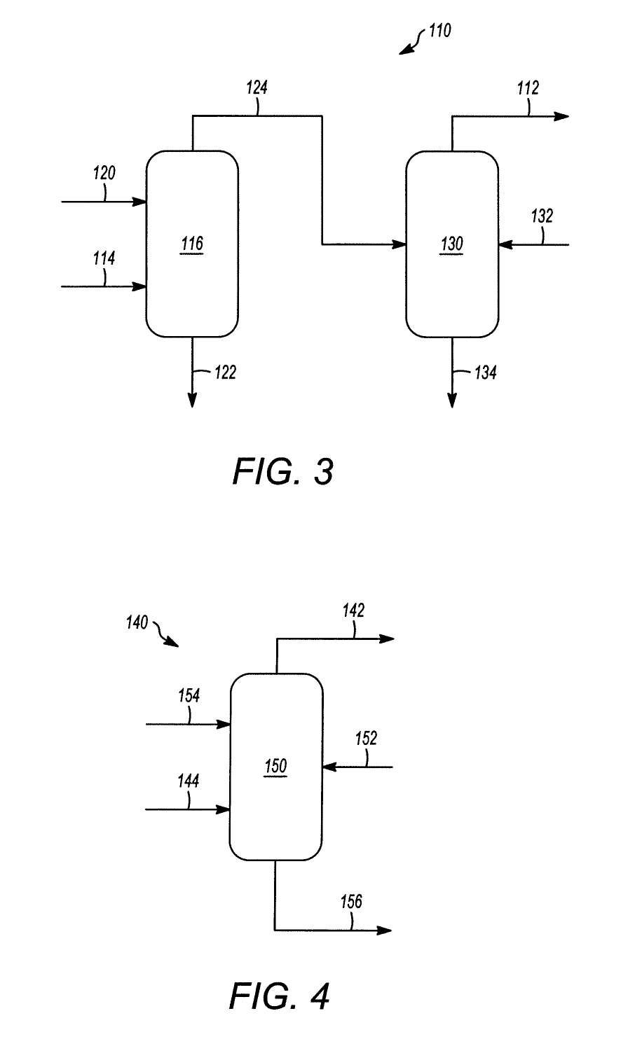

As described in greater detail below (see FIGS. 3 and 4, respectively, for example), such a mixed C₄+ hydrocarbon stream may result through processing involving washing a corresponding precursor stream with a solvent mixture of methanol and water to recover oxygenates therefrom.

The mixed C₄+ hydrocarbon stream 42 can be introduced into an olefin cracking reactor section 44 wherein at least a portion of the C₄ and C₅ olefin hydrocarbon products are cracked to form a cracked olefin effluent, shown as the line 46, comprising C₂ and C₃ olefins. The cracked olefin effluent from the line 46, if desired but not shown, can be appropriately introduced into the treatment and hydrocarbon recovery zone 30 wherein selected cracked olefin effluent species can be appropriately separated, such as in a manner known in the art.

FIG. 1 schematically illustrates that introduction of a stream of a solvent mixture comprising at least methanol and water via a line 50 into the treatment and hydrocarbon recovery zone 30. A stream (shown as the line 52), such as generally composed of methanol, water and recovered oxygenates and such as may contain some residual olefins, exits from the treatment and hydrocarbon recovery zone 30. As shown, the line 52 may desirably be introduced into a processing section 54, such as in the form of a fractionator, for example, wherein at least a portion of the water (signified by the line 56) can be separated therefrom and at least a portion of the previously absorbed oxygenates and methanol as well as residual olefins (signified by the line 60) can be returned or introduced to the oxygenate conversion reactor section 14 for contact with the oxygenate conversion catalyst and at reaction conditions effective to convert at least a portion of the oxygenates to oxygenate conversion products.

A further benefit to recovery and recycle of oxygenates can be the use of such oxygenates to pre-coke the oxygenate conversion catalyst. Such pre-coking has been found to increase the selectivity of oxygenate conversion catalysts for light olefins. Such pre-coking can be accomplished by contacting freshly regenerated oxygenate conversion catalyst with the returned or recycled oxygenate stream 60.

Those skilled in the art and guided by the teachings herein provided will appreciate that various processing systems or schemes are available and useable to effect the above-described separation in the processing section 54 and thus the broader practice of the invention is not necessarily limited to fractionation or another specific or particular such processing systems or schemes.

Turning now to FIG. 2, there is generally illustrated an embodiment of a treatment and hydrocarbon recovery zone, generally designated by the reference numeral 70, and such as for use in or as part of an olefin production scheme, such as the olefin production scheme 10 described above. The treatment and hydrocarbon recovery zone 70 desirably includes one or more unit operations whereby an oxygenate conversion product stream can be treated with a methanol and water solvent mixture to desirably remove and preferably recover selected species, such as oxygenates, such as DME.

More specifically, a compressed oxygenate conversion effluent stream or, in accordance with a preferred embodiment, at least a liquid portion thereof, here designated by the reference numeral 72 is introduced into an oxygenate recovery section 74, such as may include at least one absorber column and one or more appropriately selected washer columns. In the oxygenate recovery section 74, a stream 76 of an appropriate solvent mixture of methanol and water, such as described above, is desirably introduced and oxygenates such as methanol, dimethyl ether (DME) and other trace oxygenates including carbonyls such as acetaldehyde are absorbed therein or otherwise separated from the hydrocarbon product materials.

Thus, the oxygenate recovery section 74 forms or results in an oxygenate-rich methanol/water stream, shown as a line 80, such as comprises such oxygenate materials in methanol/water and such as may contain some residual olefins, and a stream such as comprises such hydrocarbon product materials, shown as a line 82. The oxygenate-rich methanol/water stream of line 80 can, if desired, be treated in a manner such as known in the art such as described above relative to the stream shown as the line 52 in FIG. 1, for example.

The hydrocarbon product material stream of line 82, if desired and as described above, can be further processed such as by being introduced into an caustic wash section 84 wherein such hydrocarbon product material can be processed such as by being conventionally washed with a caustic solution (not shown) to neutralize any acid gases and dried prior to passage of a resulting treated stream 86 onto a desired gas concentration and product recovery system 90. Gas concentration and product recovery systems such as used for the processing of the effluent resulting from such oxygenate conversion processing are well known to those skilled in the art and do not generally form limitations on the broader practice of the invention as those skilled in the art and guided by the teachings herein provided will appreciate.

In the gas concentration and product recovery system 90, the remaining hydrocarbon product material can be processed such as to form desired hydrocarbon fraction streams. For example, and consistent with the processing scheme shown in FIG. 1 the gas concentration and product recovery system 90 may desirably form a fuel gas stream 92, an ethylene stream 94, a propylene stream 96 and a mixed C₄+ hydrocarbon stream 100, such as generally composed of butylene and heavier hydrocarbons.

Turning now to FIG. 3, there is more specifically illustrated a processing scheme in accordance with one embodiment and here generally designated by the reference numeral 110. In the processing scheme 110, a mixed C₄+ hydrocarbon stream, such as described above and here shown as the line 112, results through processing involving washing a corresponding oxygenate-rich C₄+ hydrocarbon stream, shown as the line 114, with a solvent mixture of methanol and water to recover oxygenates therefrom.

As shown in FIG. 3, the oxygenate-rich C₄+ hydrocarbon stream from the line 114 is introduced into a lower portion of a first wash column 116. A solvent mixture, shown as the line 120 and comprising at least methanol and water, is introduced into an upper portion of the wash column 116. Thus, such a first wash column is sometimes hereinafter referred to as a methanol/water wash column. In the methanol/water wash column 116, oxygenates such as dimethyl ether (DME) and other trace oxygenates including carbonyls such as acetaldehyde are absorbed in or otherwise effectively removed via the methanol/water solvent mixture and thus are separated from the C₄+ hydrocarbon product materials.

Exiting from a lower portion of the methanol/water wash column 116 is a line 122 generally composed of a stream of oxygenate-rich methanol/water and such as may contain some residual olefins. Such an oxygenate-rich methanol/water stream can, if desired, be treated in a manner such as known in the art such as described above relative to the stream shown as the line 52 in FIG. 1, for example. Alternatively, a sieve drier, a stripper column or some alternative device or apparatus can be employed, if desired, to effect desired removal of water therefrom prior to return or recycle thereof or component portions therefrom to an associate oxygenate conversion reactor.

Exiting from an upper portion of the methanol/water wash column 116 is a line 124 generally composed of a stream containing the methanol/water washed C₄+ hydrocarbons. Those skilled in the art and guided by the teaching herein provided will appreciate that the line 124 generally composed of the stream containing the washed C₄+ hydrocarbons may also contain some residual amount of oxygenates such as residual amounts of DME and the like. In addition, such a line 124 may also contain methanol such as carried over with the C₄+ hydrocarbon. To reduce and preferably minimize methanol carryover to downstream processing units and also remove oxygenates such as may have been carried over from the first or methanol/water wash column 116, the processing scheme 110 desirably also includes a second or follow-up wash column, designated by the reference numeral 130.

As shown in FIG. 3, the line 124 generally composed of the stream containing the washed C₄+ hydrocarbons can desirably introduced into a lower portion of the second wash column 130. A solvent comprising and, in accordance with at least certain preferred embodiments, consisting essentially of wash water is, introduced via a line 132 into an upper portion of the second wash column 130. Thus, such a second wash column is sometimes hereinafter referred to as a water wash column. In the water wash column 130, residual oxygenates and carryover methanol are desirably washed or otherwise effectively removed from the processed material such as to form the line 112 composed of a C₄+ hydrocarbon stream having an appropriately reduced oxygenate content and a line 134 composed of a water stream containing removed oxygenates and carryover methanol.

In accordance with certain preferred embodiments, such C₄+ hydrocarbon streams having a reduced oxygenate content generally desirably have an oxygenate content of less than 1500 ppmw equivalent water based on oxygen. In accordance with other certain preferred embodiments, C₄+ hydrocarbon streams having a reduced oxygenate content have an oxygenate content of less than 1000 ppmw equivalent water based on oxygen and more preferably have an oxygenate content of less than 650 ppmw equivalent water based on oxygen.

Those skilled in the art and guided by the teachings herein provided will appreciate that such a resulting washed C₄+ stream materials can, in specific embodiments, be appropriately processed through a drying or other water removal process such as via a stripper column or a dryer system, such as to appropriately reduce the water content thereof.

The C₄+ hydrocarbon stream having an appropriately reduced oxygenate content can be desirably processed, as described above, through an olefin cracking reactor section wherein at least a portion of the C₄ and C₅ olefin hydrocarbon products can desirably be cracked to form additional light olefins products. As will be appreciated, and in accordance with certain preferred embodiments, such olefin cracking processing can desirably be practiced after the C₄+ hydrocarbon stream has been appropriately processed through a drying or other water removal process, as described above.

The water stream containing removed oxygenates and carryover methanol of line 134 can be appropriately treated, such as in a manner known in the art, to permit appropriate recovery, recycle or disposal of the component portions thereof. For example, a sieve drier, a stripper column or some alternative device or apparatus can be employed, if desired, to effect desired removal of water therefrom prior to return or recycle of removed oxygenates and/or methanol therefrom to an associated oxygenate conversion reactor.

Through the inclusion of such a second wash column, such a processing may advantageously better ensure reduced or minimal methanol carryover with the C₄+ materials and thus reduce or minimize possible impact thereof on downstream processing. In addition, such inclusion of such a second wash column can better ensure oxygenate removal by affording a further opportunity remove oxygenates such as may have remained in the process stream after treatment in the first wash column.

Turning now to FIG. 4, there is more specifically illustrated a processing scheme, generally designated by the reference numeral 140, in accordance with another embodiment, wherein a mixed C₄+ hydrocarbon stream, such as described above and shown as the line 142, results through processing involving washing a corresponding oxygenate-rich C₄+ hydrocarbon stream with a solvent mixture of methanol and water to recover oxygenates therefrom. The processing scheme 140 differs primarily from the processing scheme 110 described above in that the processing scheme 140 combines methanol/water washing and water washing in a single column.

As shown in FIG. 4, an oxygenate-rich C₄+ hydrocarbon stream, shown as a line 144, is introduced into a lower portion of a wash column 150. A solvent mixture comprising at least methanol and water is introduced via a line 152 into an intermediate portion of the wash column 150. Oxygenates such as dimethyl ether (DME) and other trace oxygenates including carbonyls such as acetaldehyde are absorbed in the methanol/water solvent mixture and thus are separated from the C₄+ hydrocarbon product materials.

The methanol/water washed C₄+ hydrocarbon product materials advance towards the upper portion of the wash column 150, whereat a solvent or wash fluid comprising and, in accordance with at least certain preferred embodiments, consisting essentially of wash water is introduced via a line 154. Such wash water is desirably effective to wash or otherwise effectively remove residual oxygenates and carryover methanol from the processed material such as to form the line 142 composed of an oxygenate-free C₄+ hydrocarbon stream.

Exiting from a lower portion of the wash column 150 is a line 156 generally composed of a stream of oxygenate-rich methanol/water and such as may contain some residual olefins. Such an oxygenate-rich methanol/water stream can, if desired, be treated in a manner such as known in the art such as described above relative to the stream shown as the line 52 in FIG. 1, for example.

Such a single wash column processing scheme can advantageously reduce equipment costs, as compared to the above-described multiple wash column processing scheme.

While embodiments have been described above involving processing, e.g., washing with a solvent mixture of methanol and water in accordance herewith, an oxygenate-rich C₄+ hydrocarbon stream, those skilled in the art and guided by the teaching herein provided will appreciate that the broader practice of the invention is not necessarily so limited. For example, such washing with a solvent mixture of methanol and water can, if desired and in accordance with certain alternative embodiments, be applied to selected portions of such oxygenate-rich hydrocarbon streams.

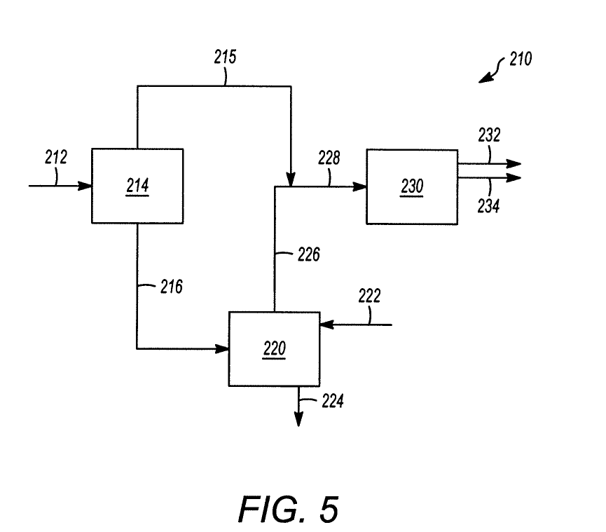

Thus, in accordance with one such embodiment, such an oxygenate-rich C₄+ hydrocarbon stream is separated or split, into a lighter portion (C₄) and a heavier portion (C₅+) wherein the heavier oxygenates would concentrate in the heavier portion. As described in greater detail below, such stream separation and subsequent processing can desirably serve to reduce downstream processing loads and requirements. A processing scheme in accordance with one such embodiment is illustrated in FIG. 5 and generally designated by the reference numeral 210. In the processing scheme 210, an oxygenate-rich C₄+ hydrocarbon stream, shown as the line 212, and such as resulting from an oxygenate conversion reactor section such as described above and not here shown, is introduced into an appropriate splitter processing device or apparatus 214 to effect such a process split. In accordance with specific embodiments, suitable such processing devices to effect such a process split may include a fractionation column, a flash drum or the like.

The splitter device 214 desirably forms, produces or results in a stream, shown as the line 215, composed of lighter carbon-containing materials (e.g., C₄ materials) and in which lighter oxygenates such as DME and/or water concentrate and a stream, shown as the line 216, composed of heavier carbon-containing materials (e.g., C₅+ materials) and in which the heavier oxygenates concentrate. As shown, the stream line 216 can be appropriately introduced into an oxygenate recovery section 220 such as comprising a methanol/water wash column. In the oxygenate recovery section 220, a stream 222 of an appropriate solvent mixture of methanol and water, such as described above, is desirably introduced and heavier oxygenates are absorbed therein or otherwise separated from the C₅+ hydrocarbon product materials. Thus, the oxygenate recovery section 220 forms or results in an oxygenate-rich methanol/water stream, shown as a line 224, such as comprises such oxygenate materials in methanol/water and such as may contain some residual olefins and a stream such as comprises such washed C₅+ hydrocarbon product materials, shown as line 226.

Those skilled in the art and guided by the teachings herein provided will appreciate that by limiting the process stream materials subject to methanol/water solvent mixture processing as herein described, the size of the corresponding process apparatus or device, e.g., absorber or wash column, can be correspondingly reduced or minimized.

The stream line 215, largely composed of C₄ materials and such as may contain lighter oxygenates and water, and the stream line of washed C₅+ materials 226 can, if desired, be combined such as to form a stream 228 and subjected to further processing such as by being introduced into a light oxygenate and/or water removal section 230 such as comprising a stripper column or the like device such as to effect removal of light oxygenates and/or water from such process stream materials. Thus, the light oxygenate and/or water removal section 230 produces or results in a mixed C₄+ hydrocarbon stream, shown as a 232, and a stream containing separated light oxygenates and/water, shown as a line 234. The mixed C₄+ hydrocarbon stream line 232 can be introduced into an olefin cracking reactor section (not shown), as described above, wherein at least a portion of the C₄ and C₅ olefin hydrocarbon products are cracked to form a cracked olefin effluent. The stream line 234 containing separated light oxygenates and/water can be appropriately treated, such as in a manner known in the art, to permit appropriate recovery, recycle or disposal of the component portions thereof. For example, the stream line 234 or selected portions thereof can desirably be recycled back to the oxygenate conversion reactor for desired processing.

In accordance with certain embodiments desirable methanol and water combinations or mixtures for appropriately balancing maximizing oxygenate extraction while minimizing C₄+ hydrocarbon extraction generally include between 5 and 80 percent by weight methanol. Correspondingly, desirable methanol and water combinations or mixtures for appropriately balancing maximizing oxygenate extraction while minimizing C₄+ hydrocarbon extraction generally include between 20 and 95 percent by weight water.

In accordance with certain preferred embodiments, such C₄+ hydrocarbon streams having a reduced oxygenate content generally desirably have an oxygenate content of less than 800 ppmw equivalent water based on oxygen. In accordance with other certain preferred embodiments, C₄+ hydrocarbon streams having a reduced oxygenate content preferably have an oxygenate content of less than 650 ppmw equivalent water based on oxygen.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

### Example 1 and Comparative Example 1

In these tests, a solvent mixture of 80% methanol and 20% water (Example 1-80% MeOH/20% H₂O) was tested and compared with application of a solvent composed of 100% methanol (Comparative Example 1) in a vapor-liquid adsorption contact with a specified C₄+ stream. The contacting was conducted at conditions including a temperature of 50° C. and a pressure of 250 psig (18 bar absolute). The respective feed compositions are shown in TABLE 1, below, and are shown on a water free basis, meaning that the feed compositions have been normalized to 100% without water being present. The results of the testing are provided in TABLE 2, below.

TABLE 1
Feed Composition

| Component | Comp. Example 1 | Comp. Example 1 |
|---|---|---|
| MeOH | 0.02 | 0.05 |
| DME | 0.05 | 0.35 |
| C1–C3 par | 2.84 | 1.88 |
| C2= | 40.9 | 36.6 |
| C3= | 34.9 | 37.4 |
| C4s | 12.2 | 13.7 |
| C5s | 5.4 | 6.1 |
| C5+ | 3.3 | 3.5 |
| Acetone | 0.25 | 0.25 |
| MEK | 0.1 | 0.08 |
| Acetaldehyde | 0.058 | 0.07 |

TABLE 2
Percent of Component Extracted

| Component | Comp. Example 1 | Comp. Example 1 |
|---|---|---|
| C4s | 80 | 20 |
| C5s | 82 | 28 |
| C5+ | 100 | 46 |
| Acetone | 92 | 93 |
| MEK | 95 | 94 |
| Acetaldehyde | 95 | 96 |

### Discussion of Results

These tests show that the use of 100% methanol (Comp. Ex. 1) resulted in relatively large amounts of C₄+ hydrocarbons being extracted into the methanol. Thus, leading to undesirable recycle to the oxygenate conversion reactor section.

In contrast, the extraction of C₄+ hydrocarbons was significantly reduced through the application of a solvent mixture composed of 80% methanol and 20% water (Example 1). Moreover, the extraction of oxygenates was largely unaffected by the use of the methanol/water solvent mixture as compared to the use of methanol alone.

### Examples 2-6 and Comparative Example 2

In these tests, the solubility of hexene in specified solvent mixtures of methanol and water, in accordance with certain embodiments (EX 2-6), and methanol (Comparative Example or “CE” 2), respectively, were evaluated. The results are shown in TABLE 3, below.

TABLE 3

| | CE 2 | EX 2 | EX 3 | EX 4 | EX 5 | EX 6 |
|---|---|---|---|---|---|---|
| Methanol/water | 100/0 | 96/4 | 90/10 | 80/20 | 70/30 | 50/50 |
| content | | | | | | |
| Hexene in | Completely | 18.6 | 5.6 | 1.3 | 0.4 | 0 |
| solution | miscible | | | | | |

### Discussion of Results

These tests show how much hexene was dissolved in each of the respective solvent/solvent mixtures. The data shows that as the water content was increased, less hexene was present in the solution. In view thereof, the use a mixture of water/methanol, with an increased relative amount of water can desirably reduce the amount of hexene that is absorbed. Consequently, in a process scheme such as described above for producing light olefins, it will be appreciated that the utilization of solvent mixtures of methanol and water can advantageously reduce the amount or proportion of olefin (i.e., hexene) that will be absorbed and recycled.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A process for producing light olefins, said process comprising:
contacting a methanol-containing feedstock in an oxygenate conversion reactor with an oxygenate conversion catalyst and at reaction conditions effective to convert the methanol-containing feedstock to an oxygenate conversion product stream comprising light olefins, C₄+ hydrocarbons and oxygenated hydrocarbons;
contacting at least a liquid portion of the oxygenate conversion product stream in an absorber with a solvent mixture comprising at least methanol and water, the solvent mixture effective to absorb a significant portion of the oxygenates from the contacted portion of the oxygenate conversion product stream; and
feeding at least a portion of the oxygenates absorbed from the contacted portion of the oxygenate conversion product stream to the oxygenate conversion reactor for contact with the oxygenate conversion catalyst and at reaction conditions effective to convert at least a portion of the oxygenates to oxygenate conversion products.

2. The process of claim 1 wherein the oxygenate conversion reactor is a fluidized bed reactor.

3. The process of claim 1 wherein:
the oxygenate conversion product stream comprises the oxygenate dimethyl ether;
said contacting of at least a portion of the oxygenate conversion product stream in an absorber with a solvent mixture comprising at least methanol and water is effective to absorb and recover a significant portion of the dimethyl ether from the oxygenate conversion product stream; and
said feeding step comprises introducing at least a portion of the dimethyl ether absorbed from the conversion product stream into the oxygenate conversion reactor.

4. The process of claim 3 wherein water is removed from recovered dimethyl ether prior to said feeding step.

5. The process of claim 1 wherein the solvent mixture consists essentially of methanol and water.

6. The process of claim 1 additionally comprising:
treating the oxygenate conversion product stream in a gas concentration system to recover light olefins and to form a C₄+ hydrocarbon stream also containing oxygenates, wherein the C₄+ hydrocarbon stream also containing oxygenates comprises the portion of the oxygenate conversion product stream contacting with the solvent mixture in the absorber and wherein as a result of said contacting a C₄+ hydrocarbon stream having a reduced oxygenate content is formed; and
contacting at least a portion of the C₄+ hydrocarbon stream having a reduced oxygenate content in an olefin cracking reactor with an olefin cracking catalyst and at reaction conditions effective to convert C₄ and C₅ olefins therein contained to a cracked olefins effluent stream comprising light olefins.

7. The process of claim 6 wherein the C₄+ hydrocarbon stream having a reduced oxygenate content has an oxygenate content of less than 1500 ppmw equivalent water based on oxygen.

8. The process of claim 7 wherein the C₄+ hydrocarbon stream having a reduced oxygenate content has an oxygenate content of less than 650 ppmw equivalent water based on oxygen.

9. The process of claim 6 wherein the solvent mixture comprises between 5 and 80 percent by weight methanol.

10. The process of claim 9 wherein the solvent mixture comprises between 20 and 95 percent by weight water.

11. The process of claim 6 wherein the solvent mixture consists essentially of methanol and water.

12. A process for producing light olefins, said process comprising:
contacting a methanol-containing feedstock in an oxygenate conversion reactor with an oxygenate conversion catalyst and at reaction conditions effective to convert the methanol-containing feedstock to an oxygenate conversion product stream comprising light olefins, C₄+ hydrocarbons and oxygenated hydrocarbons;
contacting at least a portion of the oxygenate conversion product stream in an absorber with a solvent mixture comprising at least methanol and water, the solvent mixture effective to absorb a significant portion of the oxygenates from the contacted portion of the oxygenate conversion product stream;
feeding at least a portion of the oxygenates absorbed from the contacted portion of the oxygenate conversion product stream to the oxygenate conversion reactor for contact with the oxygenate conversion catalyst and at reaction conditions effective to convert at least a portion of the oxygenates to oxygenate conversion products;
treating the oxygenate conversion product stream in a hydrocarbon recovery system to recover light olefins and to form a C₄+ hydrocarbon stream also containing oxygenates, wherein the C₄+ hydrocarbon stream also containing oxygenates comprises the portion of the oxygenate conversion product stream contacting with the solvent mixture in the absorber and wherein as a result of said contacting a C₄+ hydrocarbon stream having a reduced oxygenate content is formed; and
contacting at least a portion of the C₄+ hydrocarbon stream having a reduced oxygenate content in an olefin cracking reactor with an olefin cracking catalyst and at reaction conditions effective to convert C₄ and C₅ olefins therein contained to a cracked olefins effluent stream comprising light olefins.

13. The process of claim 12 wherein the solvent mixture comprises between 5 and 80 percent by weight methanol and between 20 and 95 percent by weight water.

14. A system for converting methanol to light olefins, said system comprising:
a reactor for contacting a methanol-containing feedstream with catalyst and converting the methanol-containing feedstream to an oxygenate conversion product stream comprising light olefins, C₄+ hydrocarbons and oxygenates;
an absorber wherein at least a liquid portion of the oxygenate conversion product stream contacts a solvent mixture comprising at least methanol and water, the solvent mixture effective to absorb a significant portion of the oxygenates from the contacted portion of the oxygenate conversion product stream; and
a first return line wherein at least a portion of the oxygenates absorbed from the contacted portion of the oxygenate conversion product stream is introduced to the oxygenate conversion reactor for contact with the oxygenate conversion catalyst and at reaction conditions effective to convert at least a portion of the oxygenates to oxygenate conversion products.

15. The system of claim 14 wherein the oxygenate conversion reactor is a fluidized bed reactor.

16. The system of claim 14 additionally comprising a stripper whereby water is stripped from absorbed oxygenates prior to the introduction of at least a portion of the absorbed oxygenates into the oxygenate conversion reactor.

17. The system of claim 14 additionally comprising:
a gas concentration system to treat the oxygenate conversion product stream to recover light olefins and to form a C₄+ hydrocarbon stream also containing oxygenates, wherein the C₄+ hydrocarbon stream also containing oxygenates comprises the portion of the oxygenate conversion product stream contacting with the solvent mixture in the absorber and wherein as a result of the contacting a C₄+ hydrocarbon stream having a reduced oxygenate content is formed; and
a reactor for contacting at least a portion of the C₄+ hydrocarbon stream having a reduced oxygenate content with an olefin cracking catalyst and at reaction conditions effective to convert C₄ and C₅ olefins therein contained to a cracked olefins effluent stream comprising light olefins.

18. The system of claim 17 comprising:
a first wash column wherein the C₄+ hydrocarbon stream also containing oxygenates contacts the solvent mixture comprising at least methanol and water to form a first washed effluent; and
a second wash column wherein the first washed effluent is contacted with wash water to form a second washed effluent with an oxygenate content of less than 1500 ppmw equivalent water based on oxygen.

19. The system of claim 18 wherein the second washed effluent has an oxygenate content of less than 650 ppmw equivalent water based on oxygen.

20. The system of claim 17 comprising a wash column wherein the C₄+ hydrocarbon stream also containing oxygenates first contacts the solvent mixture comprising at least methanol and water to form a first wash stream and subsequently the first washed stream contacts wash water to form a washed effluent with an oxygenate content of less than 1000 ppmw equivalent water based on oxygen.
